(19) United States
(12) Patent Application Publication
(10) Pub. No.: US 2005/0010097 A1
(43) Pub. Date: Jan. 13, 2005

(54) System and method for measuring fluid volumes in brain images

(76) Inventor: Harvey E. Cline, Schenectady, NY (US)

Correspondence Address: Raymond E. Farrell, Esq.;Carter, DeLuca, Farrell & Schmidt, LLP, Suite 225, 445 Broad Hollow Road, Melville, NY, 11747, US

(21) Appl. No.: 10/608,842

(22) Filed: Jun. 26, 2003

(51) Int. Cl. A61B 5/05

(52) U.S. Cl. 600/407; 600/410

(57) ABSTRACT

A method is provided for detecting a compromised condition of a patient including the steps of (a) providing for imaging in three dimensions a region of interest; (b) providing for generating a set of 3-D image data corresponding to the imaging; (c) providing for processing the set of 3-D image data for determining a first volume of an imaged first structure within the region of interest, wherein the volume of the first structure does not change substantially during adulthood over a time interval selected from the group consisting of months and years; (d) providing for processing the set of 3-D image data for determining a second volume of an imaged second structure within the region of interest, wherein a substantial change during adulthood of the volume of the second structure over a time interval selected from the group consisting of months and years is indicative of the compromised condition; and (e) providing for calculating a ratio of the second volume to the first volume.

### FIELD OF THE INVENTION

The present disclosure relates to image processing, and particularly to measuring volumes in brain images.

### BACKGROUND OF THE INVENTION

It is well known that brain atrophy is correlated with the progression of dementia, and more particularly with Alzheimer's disease. Considerable precision is needed for tracking brain atrophy for distinguishing atypical brain atrophy from typical atrophy of a healthy brain, which is typically 3.5% per decade. Studies have shown that changes in volume of the lateral ventricles during adulthood is an indication of dementia, and more specifically, of Alzheimer's disease.

A 3-D imaging device is typically used for imaging the head region of a patient for generating image data that corresponds to the imaged region. The image data is processed, such as by segmentation and/or registration techniques for assigning a gray scale value (or color) to each data element of the image data, so that different types of tissues are assigned different values, such as gray scale value or color value. Each type of material in the data is assigned a specific value and, therefore, each occurrence of that material has the value.

Furthermore, techniques are known for differentiating between different structures or areas having the same tissue type for assigning each a specific value. An image is generated for display in accordance with the assigned values, where different tissue types are distinguishable because of the value assignment. For example, all occurrences of bone in a particular image may appear in a particular shade of light gray. This standard of coloring allows the individual viewing the image to easily understand the objects being represented in the images. Furthermore, the differentiation of tissue type, and or area or structure within a tissue type, allows for further processing, such as calculation of a volume of a desired tissue type, or area or structure within the tissue type.

The 3-D imaging device can be selected from a number of medical imaging devices known in the art for generating 3-D images, such as devices using technologies including magnetic resonance (MR), computer tomography (CT), positron emission tomography (PET), nuclear magnetic resonance (NMR), single-photon emission computed tomography (SPECT), etc. A variety of techniques are known for processing the image data, including obtaining volume measurements for desired structures.

A single volume measurement of lateral ventricles of a patient performed at a single imaging session does not typically provide useful information, as the normal size of the lateral ventricles varies for different individuals. A first measurement is used as a baseline measurement, after which subsequent images and measurements are obtained and calculated over time at time intervals such as months or years.

When using 3-D imaging to detect Alzheimer's disease in a patient by determining volume changes over time of lateral ventricles in the patient, inconsistencies in the imaging and/or image data processing techniques may interfere with accurately monitoring changes over time. Images and measurements taken at different time intervals may be performed using different machinery, a same machine that has been adjusted, reconfigured and/or upgraded and/or different or upgraded algorithms for processing data. Furthermore, a degree of manual intervention is typically used during image processing, such as for segmentation. Manual intervention is affected by subjectivity, particularly when performed at different time intervals by different people.

Accordingly, there is a need for a system and method for obtaining reliable measurements of volumes of body structures indicative of a condition or disease in which a single imaging and measurement procedure provides meaningful information and in which inconsistencies in imaging and measurement procedures taken at different time intervals-are minimized.

### BRIEF DESCRIPTION OF THE INVENTION

In one aspect of the invention a method is provided for detecting a compromised condition of a patient including the steps of (a) providing for imaging in three dimensions a region of interest; (b) providing for generating a set of 3-D image data corresponding to the imaging; (c) providing for processing the set of 3-D image data for determining a first volume of an imaged first structure within the region of interest, wherein the volume of the first structure does not change substantially during adulthood over a time interval selected from the group consisting of months and years; (d) providing for processing the set of 3-D image data for determining a second volume of an imaged second structure within the region of interest, wherein a substantial change during adulthood of the volume of the second structure over a time interval selected from the group consisting of months and years is indicative of the compromised condition; and (e) providing for calculating a ratio of the second volume to the first volume.

Steps of the method of the invention may be implemented by executing instructions on a processor, where the instructions are stored on a computer readable medium or included in a computer data signal embodied in a transmission medium.

In another aspect of the invention a computer apparatus is provided for detecting a compromised condition of a brain including means for receiving a set of 3-D image data corresponding to imaging in three dimensions of a region of interest; first means for processing the set of 3-D image data for determining a volume of an imaged first structure within the region of interest, wherein the volume of the first structure does not change substantially during adulthood over a time interval selected from the group consisting of months and years; second means for processing the set of 3-D image data for determining a volume of an imaged second structure within the region of interest, wherein a substantial change during adulthood of the volume of the second structure over a time interval selected from the group consisting of months and years is indicative of the compromised condition; and means for calculating a ratio of the volume of the second structure to the volume of the first structure.

### BRIEF DESCRIPTION OF THE DRAWINGS

FIG. 1 is a block diagram of a 3-D imaging system; and

FIG. 2 is a flowchart of a method of an aspect of the invention.

### DESCRIPTION OF THE PREFERRED EMBODIMENT

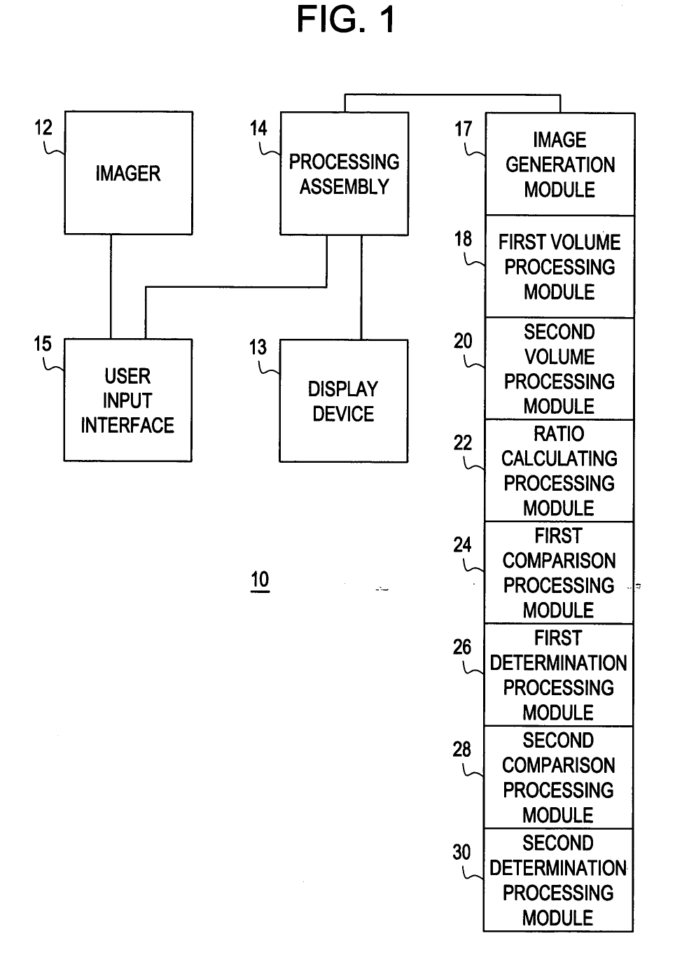

Referring to FIG. 1, a 3-D imaging system 10 is shown including an imager 12 and a processor assembly 14, where the imager 12 images a region of interest within a patient in an imaging session (also referred to as a scan), and generates image data that corresponds to the imaged region of interest. The system 10 further includes a display device 13 for displaying an image that corresponds to the image data and a user input interface 15 for allowing a user to enter information, such as data and requests, to the processing assembly 14 and/or the imager 12. The processing assembly 14 receives and processes the generated image data for generating a displayable image, differentiating between different types of tissue within the imaged region of interest, determining the volume of a first structure that is relatively static and of a second structure that is dynamic and changes over time. The processor compares the volumes of the first and second structures for assessing a specific condition of the patient. The volume measurement for the static first structure provides a reference point that is personalized for the patient.

The comparison may be performed by calculating a ratio of the volume of the second structure to the volume of the first structure. The comparison value, i.e., the ratio, provides meaningful information for a one time imaging and measurement procedure. Furthermore, for studies for detecting change over a period of time by performing imaging and measurement procedures at time intervals, such as months or years, volume inconsistencies due to inconsistencies in the imaging and measurement procedures are cancelled out and minimized by using the ratio values.

In the current example, the region of interest is within the head region of the patient, where specifically the first structure is the intracranial volume. The intracranial volume is the tissue within the skull, not including the skull. The volume of the first structure is defined by a static structure (i.e., the skull), which is typically a non-fluid structure. The volume of the first structure is static, i.e., does not substantially change in volume over a relatively long period of time, such as months or years, and more specifically does not change substantially during adulthood. A study of five patients and five healthy age matched volunteers was performed over three years. 40 three-dimensional dual echo data sets were acquired of the head in both normal patients and patients with dementia and segmented with the active contours method described below. The intracranial volume was found to be constant with only up to about a 1 percent variation in respective patients measured at different times.

The second structure is a structure that is dynamic, i.e., its volume changes over time, so that the change in volume is sufficiently substantial to be reliably detected over a period of time, such as months or years. The second structure is a dynamic structure, which in the present example is formed of fluid tissue, and more particularly, the second structure is at least one of the lateral ventricles located within the intracranial volume. Studies have shown that changes in volume of the lateral ventricles during adulthood is an indication of dementia, and more specifically, of Alzheimer's disease.

The imager 12 is an imaging assembly capable of imaging in 3-dimensions for generating image data that corresponds to at least one 3-D image. The imager 12 is further capable of generating well contrasted image data, including image data that corresponds to the imaging of fluids and non-fluids. The at least one 3-D images corresponding to the image data generated by the imager 12 includes images that collectively are well contrasted for imaged fluid matter as well as for non-fluid matter.

The imager 12 can be selected from a number of medical imaging devices known in the art for generating 3-D images, such as devices using technologies including magnetic resonance (MR), computer tomography (CT), positron emission tomography (PET), nuclear magnetic resonance (NMR), spectography positron emission computer tomography (SPECT), etc. Where the imager is an MR imager it uses at least two echoes, herein referred to as a dual echo MR imager, since the dual echo MR imager uses the at least dual echoes to obtain good contrast of fluid and non-fluid matter.

During a MR imaging session, the patient is placed inside a strong magnetic field generated by a large magnet. Magnetized protons within the patient, such as hydrogen atoms, align with the magnetic field produced by the magnet. A particular slice of the patient is exposed to radio waves that create an oscillating magnetic field perpendicular to the main magnetic field. The slices can be taken in any plane chosen by the physician or technician performing the imaging session. The protons in the patient's body first absorb the radio waves and then emit the waves by moving out of alignment with the field. As the protons return to their original state (before excitation), diagnostic images based upon the waves emitted by the patient's body are created. Like CT image slices, MR image slices can be reconstructed to provide an overall picture of the body area of interest. Parts of the body that produce a high signal are displayed as white in an MR image, while those with the lowest signals are displayed as black. Other body parts that have varying signal intensities between high and low are displayed as some shade of gray. Acquisition parameters in MR imaging influence the tissue contrast. For example, in dual echo imaging fluid, such as cerebral spinal fluid (CSF), may have similar contrast to brain tissue in proton density weighted images using one set of image acquisition parameters, but be much brighter than brain tissue in T2 weighted images using another set of image acquisition parameters.

In dual echo MR imaging, two images may be generated for each slice, where the first and second images are generated using different image acquisition parameters, such as different repetition times (TR) and/or echo times (TE). Depending on the image acquisition parameters used for each image generated, one image may show better contrast between certain tissues of interest, such as solid tissues, e.g., white and gray matter in the brain, while another image may show better contrast between other tissues of interest, such as fluids and solids, e.g., (CSF) and gray matter.

The first 3-D image corresponds to a first echo obtained with image acquisition parameters for showing better contrast of non-fluid matter. Thus, the first echo is used to provide a well-contrasted image of the first structure, which is defined by the static structure, which provides a reference point. In one embodiment of the invention, the first 3-D image is more heavily T1 weighted. The second image corresponds to a second echo obtained with image acquisition parameters for showing better contrast of fluid matter. Thus, the second echo is used to provide a well-contrasted image of the second structure, which is the dynamic structure for which change in volume is being studied. In one embodiment of the invention, the second 3-D image is more heavily T2 weighted. The first and second images may be generated in response to one excitation, and both images correspond to substantially the same region of interest as well as to substantially the same point in time.

The processing assembly 14 includes at least one processor, such as a microprocessor, a CPU, a personal computer, a PDA, a hand-held computing device, a mainframe computer, etc. Processors of the processing assembly 14 may be in data communication with one another, such as by a network such as a LAN, WAN, intranet, internet, etc. The processing assembly 14 further includes an input port 16 for receiving the image data. A variety of software modules executable by the processing assembly 14 are accessed by the processing assembly 14, and executed thereby for processing of the image data, and for determination of the condition of the region of interest. The software modules each include a series of programmable instructions executable on the processing assembly 14. The software modules may be stored on at least one computer readable medium (e.g., RAM, floppy, CD-ROM, flash memory, hard drive, etc.) or be included in a computer data signal embodied in a transmission that is accessible by the processing assembly 14. The at least one storage medium, and/or a drive associated therewith, may be external to or included within the processing assembly 14. The means for transmitting the signal may be partially or fully external to and/or included in the processing assembly 14.

The software modules include an image generation module 17 for processing the image data and generating a 3-D image set including at least one corresponding 3-D image, a first volume processing module 18 for processing the 3-D image set for determining a volume of a static structure included in the region of interest, a second volume processing module 20 for processing the 3-D image set for determining a volume of at least one lateral ventricle included in the region of interest, and a ratio calculating processing module 22 for calculating a ratio of the volume of the lateral ventricles to the volume of the static structure.

The image generation module 17 is executed by the processor assembly 14 for processing the image data, including a first image data set including image data that corresponds to the first echo, in which the first structure is well contrasted, and a second image data set including image data that corresponds to the second echo, in which the second structure is well contrasted. The image generation module 17 includes an algorithm for classifying voxels (3-D data elements) into classes that are homogeneous with respect to certain characteristics, such as intensity, texture, etc., and assigning each class a specific value, such as a gray scale value or a color value. When displayed, an image is generated in accordance with the assigned values, where different tissue types are distinguishable because of the value assignment. This standard of coloring allows the individual viewing the image to easily understand the objects being represented in the images. Of particular interest in the present invention are the classes that correspond to the first structure from the first image data set and the second structure from the second image data set.

There are known algorithms, such as segmentation and registration algorithms, for classifying voxels, including algorithms that are manual and algorithms that are automatic and require some manual intervention. Typically, a seed voxel is placed in the image within the anatomical structure of interest and adjacent voxels are successively analyzed and identified as belonging to the same structure generally if they are adjacent to a previously identified voxel and they meet a specified attribute, such as intensity or radiological density.

In one method known as active contours, the brain is segmented using a model where the surface of the active contour (bubble) moves at a velocity that depends on curvature and diffusive flow. This involves growing a bubble constrained by image parameters such as gradients and curvature and constructing a force that stops the bubble growth. The connected volume after segmentation may include regions that are not of interest, thus requiring some user intervention. For example, manual editing may be needed to separate voxels that correspond to the scalp from voxels that correspond to the intracranial volume. Further, the connected volume may include connection through an undesired narrow region, bridge or other small structure that connects different regions that are desirably separated. Methods that require manual editing are typically tedious, and are subject to inaccuracies because of inter observer error.

In one aspect of the invention, a segmentation algorithm which requires minimal user intervention is used including the steps of generating a plurality of successive layers of spheres about a circumference of a sphere containing at least one start seed point placed within an object of interest when a plurality of respective voxels contained within the spheres exceed an selected initial threshold. The generation of the layers is repeated until no further voxels contained within an outer surface of each respective layer exceed the selected initial threshold, or until at least one stop seed placed outside the object of interest is encountered to form a segmented representation of the object of interest. The selected threshold may be adjusted in response to encountering the stop seed point.

The first volume processing module 18 is executed by the at least one processor for performing an algorithm for processing the class of the first image data set for determining a first volume of the structure that corresponds to the static first structure. The second volume processing module 20 is executed by the at least one processor for performing an algorithm to process the class of the second image data set for determining a second volume of the structure that corresponds to the dynamic second structure. The ratio calculating processing module 22 calculates the ratio of the second volume to the first volume.

In one aspect of the invention, the software modules further include a first comparison processing module 24 which is executed by the processor assembly 14 for comparing the calculated ratio to a predetermined ratio, and a first determination processing module 26 which is executed by the processor assembly 14 for determining if the compromised condition is detected in accordance with the comparing. Specifically, if the calculated ratio is outside of a first predetermined threshold range a determination is made that an indication of the presence of Alzheimer's disease exists.

In another aspect of the invention, the imager 12 performs a series of imaging sessions for imaging the region of interest at different times spaced by time intervals, such as months or years. A first and second set of image data is generated for each imaging session. For each imaging session, the image generation module 17 processes the first and second sets of data for classifying the data into classes. The first and second volume processing modules 18, 20 process classes of interest that correspond to the first and second structure for determining the respective volume of the first and second structures. The software modules further include a second comparison processing module 28 which is executed by the processor assembly 14 for comparing the calculated ratio of one imaging session to a calculated ratio of at least one previous imaging session, and a second determination processing module 30, which is executed by the processor assembly 14 for determining if the compromised condition is detected in accordance with the comparing. Specifically, if the difference between the calculated ratios corresponding to imaging sessions performed at different time intervals is outside of a second predetermined threshold range that corresponds to the time interval a determination is made that an indication of the presence of Alzheimer's disease exists.

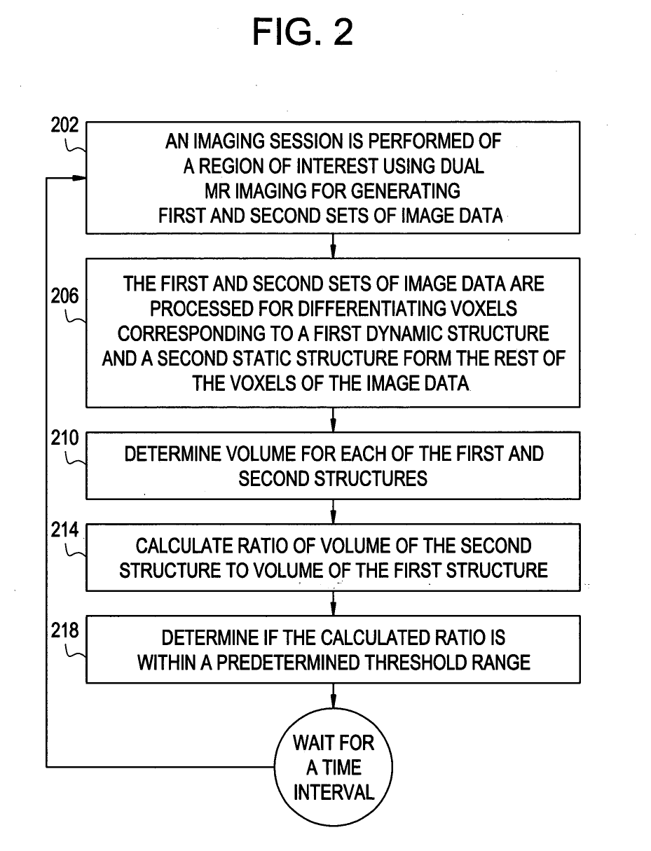

With respect to FIG. 2, a method of one aspect of the invention is shown. At step 202, an imaging session is performed of a region of interest using dual MR imaging for generating first and second sets of image data. At step 206, the first and second sets of image data are processed for differentiating voxels corresponding to a first dynamic structure and a second static structure from the rest of the voxels of the image data. At step 210, a volume is determined for each of the first and second structures. At step 214, a ratio of the volume of the second structure to the volume of the first structure is calculated. At step 218, a determination is made if the calculated ratio is within a first predetermined threshold range. Steps 202-218 are repeated after a time interval, such as months or years. The calculated ratio of the last iteration is compared to the calculated ratio of at least one previous iteration, and a determination is made if the difference is within a second predetermined threshold range in accordance with the time interval. Steps 202 through 218 are repeated as desired.

The described embodiments of the present disclosure are intended to be illustrative rather than restrictive, and are not intended to represent every embodiment of the present disclosure. Various modifications and variations can be made without departing from the spirit or scope of the present disclosure as set forth in the following claims both literally and in equivalents recognized in law.

1. A method for detecting a compromised condition of a patient comprising the steps of:
(a) providing for imaging in three dimensions a region of interest;
(b) providing for generating a set of 3-D image data corresponding to the imaging;
(c) providing for processing the set of 3-D image data for determining a first volume of an imaged first structure within the region of interest, wherein the volume of the first structure does not change substantially during adulthood over a time interval selected from the group consisting of months and years;
(d) providing for processing the set of 3-D image data for determining a second volume of an imaged second structure within the region of interest, wherein a substantial change during adulthood of the volume of the second structure over a time interval selected from the group consisting of months and years is indicative of the compromised condition; and
(e) providing for calculating a ratio of the second volume to the first volume.

2. The method according to claim 1, wherein the volume of the first structure is defined by the volume of a skull of the patient.

3. The method according to claim 1, wherein the second structure is included in the lateral ventricles of the brain of the patient.

4. The method according to claim 1, wherein the volume of the first structure is the intracranial volume of the brain of the patient.

5. The method according to claim 1, further comprising the step of:
(f) providing for determining if the calculated ratio is within a first predetermined range for detecting the compromised condition.

6. The method according to claim 1, further comprising the steps of:
(f) providing for repeating steps (a)-(e) at least one time, each repetition performed after a time interval;
(g) providing for comparing the calculated ratio of one repetition to at least one ratio calculated at a previous time interval; and
(h) providing for determining if the difference between a ratio that corresponds to one repetition and a ratio that corresponds to a previous repetition is within a second predetermined threshold for detecting the compromised condition.

7. The method according to claim 1, wherein the compromised condition is Alzheimer's disease.

8. The method according to claim 1, wherein step (a) includes providing for imaging using magnetic resonance imaging.

9. The method according to claim 1, wherein step (a) includes providing for imaging using at least dual echo magnetic resonance imaging.

10. The method according to claim 1, wherein step (a) includes the steps of:
providing for generating a first set of 3-D image data in which the first structure is well defined using a first echo of an at least dual echo MRI; and
providing for generating a second set of 3-D image data in which the second structure is well defined using a second echo of the at least dual echo MRI.

11. The method according to claim 1, wherein at least one of steps (c) and (d) includes the step of providing for automatically segmenting for determining the first and second volume, respectively.

12. The method according to claim 11, wherein in the step of providing for automatically segmenting includes the steps of:
providing for generating a plurality of successive layers of fixed radius spheres about a circumference of a sphere containing at least one seed point placed within the object of interest, including one of the first and second structures, when a plurality of respective voxels contained within the spheres exceed a selected threshold; and
providing for repeating generation of the layers until no further voxels contained within an outer surface of each respective layer exceed the selected threshold, the layers forming a segmented representation of the object of interest.

13. A computer apparatus for detecting a compromised condition of a brain comprising:
means for receiving a set of 3-D image data corresponding to imaging in three dimensions of a region of interest;
first means for processing the set of 3-D image data for determining a volume of an imaged first structure within the region of interest, wherein the volume of the first structure does not change substantially during adulthood over a time interval selected from the group consisting of months and years;
second means for processing the set of 3-D image data for determining a volume of an imaged second structure within the region of interest, wherein a substantial change during adulthood of the volume of the second structure over a time interval selected from the group consisting of months and years is indicative of the compromised condition; and
means for calculating a ratio of the volume of the second structure to the volume of the first structure.

14. The computer apparatus according to claim 13, wherein the volume of the first structure is the intracranial volume of the brain of the patient.

15. The computer apparatus according to claim 13, wherein the volume of the first structure is defined by the volume of a skull of the patient.

16. The computer apparatus according to claim 13, wherein the second structure is included in the lateral ventricles of the brain of the patient.

17. The computer apparatus according to claim 13, further comprising means for determining if the calculated ratio is within a first predetermined range for detecting the compromised condition.

18. The computer apparatus according to claim 13, wherein the means for receiving receives a series of sets of 3-D image data corresponding to repetitive imaging of the region of interest, with each repetition performed after a time interval, the first and second means for processing processes each received set of 3-D image data; the means for calculating the ratio calculates a ratio that corresponds to each received set of 3-D image data, the computer apparatus further comprising:
means for comparing a first calculated ratio corresponding to one received set of 3-D image data of the series of received sets of 3-D image data to a second calculated ratio that corresponds respectively to a preceding set of 3-D image data of the series of received sets of 3-D image data; and
means for determining if the difference between the first and second ratios is within a second predetermined threshold for detecting the compromised condition.

19. The computer apparatus according to claim 13, wherein the compromised condition is Alzheimer's disease.

20. The computer apparatus according to claim 13, wherein the received set of 3-D image data is generated using magnetic resonance imaging.

21. The computer apparatus according to claim 13, wherein the received set of 3-D image data is generated using at least dual echo magnetic resonance imaging.

22. The computer apparatus according to claim 13, wherein the received set of 3-D image data includes a first set of 3-D image data in which the first structure is well defined using a first echo of an at least dual echo MRI; and a second set of 3-D image data in which the second structure is well defined using a second echo of the at least dual echo MRI.

23. The computer apparatus according to claim 13, wherein at least one of the first and second means of processing includes means for determining the first volume and means for determining the second volume, respectively includes means for performing automatic segmentation.

24. The computer apparatus according to claim 23, wherein the means for performing automatic segmentation includes:
means for generating a plurality of successive layers of fixed radius spheres about a circumference of a sphere containing at least one seed point placed within the object of interest, including one of the first and second structures, when a plurality of respective voxels contained within the spheres exceed a selected threshold; and
means for repeating generation of the layers until no further voxels contained within an outer surface of each respective layer exceed the selected threshold, the layers forming a segmented representation of the object of interest.
